SEQUENCE LISTING (32 sequences)

SEQ ID NO: 1 - DNA, 3579 bp, Bacillus licheniformis
  CDS (1)..(3579)
ttgacaggtcaactagttcagtatggacgacaccgccagcgcagaagctatgcacgcattagcgaagtgttagaattaccaaatctcattgaaattcaaacctcttcttatcagtggtttcttgatgagggtcttagagagatgtttcaagacatatcgccaattgaggatttcactggtaacctctctctggaatttatcgactacagcttgggcgagcctaagtatccggtagaagaatcaaaagagcgggatgtgacctattcagctccgctgcgggttaaagtccgcttaatcaacaaagaaaccggcgaagtaaaggatcaggatgtcttcatgggcgatttccctattatgacagacactggaaccttcattatcaacggtgcagaacgggtcatcgtatctcagctcgttcgttctccaagtgtatattttagtggtaaagtagacaaaaacggtaagaaaggttttaccgcgactgtcattccaaaccgtggcgcatggttagaatacgagactgatgcgaaagatgttgtttacgtacgcatcgatcgcacacgtaagttgccggttacggttcttttgcgtgctcttggcttcggatctgaccaagagatcattgatctcatcggtgaaaacgagtatctgcgcaatacgcttgataaagataatacggaaaacaccgataaagcgcttcttgaaatctacgagcgtcttcgtccaggggagccgcctaccgtagaaaacgcaaaaagcctgcttgattcaaggttctttgatccgaaaagatatgaccttgcgagtgtaggacgttataaaattaataaaaagcttcacatcaaaaaccgactttttaatcagcggcttgctgaaacgctagtcgaccctgaaacaggcgaaatccttgccgaaaaaggagcgattttagacagaagaacgcttgataaagttctgccgtaccttgaaaacggaatcggttttaaaaagctttatccgaacggcggagttgtcgaagacgaagtaacgcttcagtctatcaaaatctatgctccgacagaccaagaaggggagcagacaatcaatgtgattggaaatgcttatatcgaagaaggcgttaagaatattacaccttctgatattatcgcttccatcagctatttctttaacctgcttcacggagtgggcgataccgacgatatcgaccatctaggaaaccgccgtctccgttcagtgggagagcttctgcaaaaccaattccgtattggtttaagcagaatggagcgcgttgttcgtgaaagaatgtctattcaagatacaaacacgatcacgccgcagcagctgatcaatattcgccctgtcatcgcatcaatcaaagagtttttcggaagctcgcagctttctcagtttatggatcagacgaatccgcttgctgagctgacgcataagcgccgtctgtcagcgctcggaccgggcggtttgactcgtgagcgcgccggaatggaagtccgtgacgttcactattcacactacggccggatgtgtccgattgaaacccctgagggtccaaacatcggcttgatcaactcgctttcttcattcgcgaaagtgaaccgtttcggcttcatcgaaacgccgtatcgccgcgtcgatcctgaaactggaaaagtaacgccgagaatcgattacttgacagctgatgaagaagacaactatgtcgttgcccaggcaaacgcacgcctgaatgatgacggttcttttgtggatgacagcatcgtcgcccgtttcagaggggagaacaccgttgttccgaaagaccgcgtcgactatatggacgtttcgcctaaacaggttgtctctgccgcgactgcatgtattcctttcttggaaaacgatgactcaaaccgcgcccttatgggagcgaacatgcaacgtcaggccgtacctcttatgcagcctgaatcgccgatcgtcggaaccgggatggaatatgtatctgcgaaagactccggtgccgctgttatttgccgccaccctggaatcgttgaacgggtggaagcgaagaacatctgggtgcgccgctatgaagaagtcgacggccagaaagtcaaagggaaccttgataaatacagcctgctgaagtttgtccgttccaaccagggaacttgctacaaccagcgtccgatcgtaagcgtcggtgatgaggttgaaaaaggtgaaattttagctgacggtccgtccatggaaaaaggtgagcttgcccttggacgcaacgtcatggtcggctttatgacatgggatggctacaactatgaggatgccatcatcatgagcgaacgccttgtaaaagacgacgtatacacgtctattcatattgaagaatacgaatcagaggcccgggatacaaaactcggacctgaagaaatcactcgcgatattccgaacgtcggtgaggacgctcttcgcaatctcgatgaacgcggaattatccgtgtcggtgctgaagtaaaagacggagatcttcttgttggtaaagtaacccctaaaggtgttacagagcttacggcggaagaacgcctgcttcatgccatcttcggggaaaaagcgcgtgaagtgcgtgatacgtcgctgcgtgtacctcacggaggcggcggtatcatccttgatgtaaaagtgttcaaccgcgaagacggagacgaactgcctccgggcgttaaccagctcgtccgcgtctacatcgttcagaagcgtaaaatttctgaaggggacaaaatggccggacgccacggtaacaaaggtgttatttcgaaaattcttccggaggaagatatgccgtatctgcctgacggaacgccgattgacatcatgttaaacccgctgggcgtaccatcgcgtatgaacatcgggcaggtgttggagctgcaccttggtatggctgcacgccgcctcggtctgcatgtcgcgtcacctgtatttgacggtgcccgcgaagaagatgtgtgggaaacccttgaagaagccggcatgtcaagagacgcaaaaaccgtcctttacgacggccgaactggggagccgtttgacaaccgggtttctgtcggcatcatgtacatgatcaaactggcacacatggttgacgacaaattgcacgcacgttcaaccggtccttactcactcgttacccagcagcctctcggaggtaaagcgcagttcggcggacagcgttttggagagatggaagtttgggcgcttgaagcttatggtgcagcatatacgctacaagagatcctgactgttaaatcggatgatgtcgtaggccgtgtgaaaacatatgaagccatcgtaaaaggcgacaatgttccagaacctggtgttccggaatcgttcaaagtattgatcaaagagcttcaaagcttaggtatggacgtcaaaattctatcaagcgacgaagaagaaatcgaaatgagagacttggaagacgacgaagacgcgaaacaaaacgaagggctttctctgccgaatgatgaagagtccgaagagttggtttctgctgacgcagaacgcgatgtcgttacaaaagaa

SEQ ID NO: 2 - protein, 1193 aa, Bacillus licheniformis
LTGQLVQYGRHRQRRSYARISEVLELPNLIEIQTSSYQWFLDEGLREMFQDISPIEDFTGNLSLEFIDYSLGEPKYPVEESKERDVTYSAPLRVKVRLINKETGEVKDQDVFMGDFPIMTDTGTFIINGAERVIVSQLVRSPSVYFSGKVDKNGKKGFTATVIPNRGAWLEYETDAKDVVYVRIDRTRKLPVTVLLRALGFGSDQEIIDLIGENEYLRNTLDKDNTENTDKALLEIYERLRPGEPPTVENAKSLLDSRFFDPKRYDLASVGRYKINKKLHIKNRLFNQRLAETLVDPETGEILAEKGAILDRRTLDKVLPYLENGIGFKKLYPNGGVVEDEVTLQSIKIYAPTDQEGEQTINVIGNAYIEEGVKNITPSDIIASISYFFNLLHGVGDTDDIDHLGNRRLRSVGELLQNQFRIGLSRMERVVRERMSIQDTNTITPQQLINIRPVIASIKEFFGSSQLSQFMDQTNPLAELTHKRRLSALGPGGLTRERAGMEVRDVHYSHYGRMCPIETPEGPNIGLINSLSSFAKVNRFGFIETPYRRVDPETGKVTPRIDYLTADEEDNYVVAQANARLNDDGSFVDDSIVARFRGENTVVPKDRVDYMDVSPKQVVSAATACIPFLENDDSNRALMGANMQRQAVPLMQPESPIVGTGMEYVSAKDSGAAVICRHPGIVERVEAKNIWVRRYEEVDGQKVKGNLDKYSLLKFVRSNQGTCYNQRPIVSVGDEVEKGEILADGPSMEKGELALGRNVMVGFMTWDGYNYEDAIIMSERLVKDDVYTSIHIEEYESEARDTKLGPEEITRDIPNVGEDALRNLDERGIIRVGAEVKDGDLLVGKVTPKGVTELTAEERLLHAIFGEKAREVRDTSLRVPHGGGGIILDVKVFNREDGDELPPGVNQLVRVYIVQKRKISEGDKMAGRHGNKGVISKILPEEDMPYLPDGTPIDIMLNPLGVPSRMNIGQVLELHLGMAARRLGLHVASPVFDGAREEDVWETLEEAGMSRDAKTVLYDGRTGEPFDNRVSVGIMYMIKLAHMVDDKLHARSTGPYSLVTQQPLGGKAQFGGQRFGEMEVWALEAYGAAYTLQEILTVKSDDVVGRVKTYEAIVKGDNVPEPGVPESFKVLIKELQSLGMDVKILSSDEEEIEMRDLEDDEDAKQNEGLSLPNDEESEELVSADAERDVVTKE

SEQ ID NO: 3 - DNA, 3546 bp, Bacillus clausii
  CDS (1)..(3546)
ttgacaggtcaactaattcagtatggacgtcaccgccagcggagaagctatgcgcgaattaatgaagtgctcgaactgccaaacttgattgaaattcaaacagcttcttatcaatggtttcttgatgagggtttgcgggagatgtttcaagacatttcaccgatccaagacttcactggcaatttagtgttagagtttattgattacagtctaggagaaccaaagtatcctgttgatgagtcaaaagagcgtgacgtgacgtttgccgctcctctacgtgtcaaagttcgccttattaataaagagacaggcgaagtaaaagaacaggaagtctttatgggcgatttcccgttgatgacagaaacagggacatttatcatcaatggcgctgagcgcgttatcgtttctcagcttgttctstcaccgagcgtttactatagccaaaagctcgacaaaaacggaaaaaaaggctttactgccactgtcattccgaaccgcggagcgtggcttgagcttgagacagatgcaaaagatattgtttatgtacgtattgaccgtactcgtaaaattccagtaacagtacttttgcgtgctctaggctttgggtctgatcaagaaatcgttgaccttttaggcgaaaacgagtatttgcgcaacacgcttgagaaagataatacggactcaactgataaagtgctgcttgaaatctatgagcgtttgcgcccgggcgagccgccaacagtagaaaatgcgaaaagcttgcttgaatctcgctttttcgatcctaagcgttatgacctcgcaaatgtcggtcgttataaaataaataaaaagcttcatatcaaaaatcggctatttaaccaacgtttggcggaaaagcttgttgatccggaaacaggcgaagttctagctgaagagggaacacttcttgaccgcagaacgttggataaactgatcccgcatttggagaaaaatgttggtttccgtacagcccgtacatctggcggcgttcttgaggaatccgacgtcgaaatccaatcggttaaaatttatgtagcagatgactacgagggcgagcgcgtcatttccgttattagcaacggcatggttgaacgtgacgtgaagcatattgcccctgctgatatcatcgcttccatcagctatttcttcaacttgctgcatggtgtcggcgatacagacgacattgaccatttgggcaaccgccgtctccgttcggttggggaactgttgcaaaaccaattccgaattggcctttctcggatggaacgcgtcgtccgtgaacggatgtcgattcaagacccgaatgtaattacgccacaagcgcttattaacattcgcccagtcatcgcatcgataaaagagttctttggcagctcgcagctttcacagttcatggaccaaacgaacccgctcgctgaactgacgcataagcgccgtctttccgcgcttggcccaggcggcttaactcgtgagcgcgctggaatggaagtgcgtgacgttcactactcccattacggccggatgtgtccgattgaaacgccggaaggtccgaacattggcttaattaacacgctgtcctcttatgcgaaagtgaatgagttcggctttatggaaacaccatatcgccgtgttgaccctgaaacagggaaagtgacagcgcgtatcgactatttaacagccgatgaagaggacaattatgtagttgcccaagcaaatgcgaagctgaatgaagacggatcgtttgtcgatgataacatcattgcccgcttccgcggtgaaaacaccgtcgttccttgcgaccgtgtcgactatatggacgtttcgcctaaacaagttgtctctgcggcgacgtcatgtattccgtttttggaaaatgacgactcgaaccgcgcactaatgggcgcaaacatgcagcgccaggccgtgcctttgctcgttccagaagcaccgcttgtcggaacagggatggagcacgtgtctgctaaagattcaggggctgctgttgtctcaaaatatgccggtatcgttgaacgcgttactgctaaagaaatttgggtccgccgcattgaagaagtagatggcaaagaaacgaaaggcgaccttgataaatacaaactacaaaaatttgtgcgctccaaccagggcacaagttataatcagcgcccgattgtacgcgaaggcgatcgcgttgaaaaacgtgaaatcctcgcagatggtccttcaatggagatgggcgaaatggcgcttggccgcaacgtgcttgtcgcctttatgacatgggacggctacaactacgaagatgcgatcattttaagtgagcgcctagtcaaagatgacgtttatacgtccatccatattgaggaatatgaatcggatgcccgtgatacaaaactcggacctgaagagattacgcgcgatattccgaacgttggtaagaatgcattacgcaaccttgatgagcggggaattattcgcatcggggctgaagtcaaagatggcgacattcttgttggtaaagtgacgccaaagggcgtaacggagctgacggcggaagaacgcctcttgcatgcgatttttggagagaaagcccgtgaagtgcgggatacttcattgcgtgcaccgcatggtggagacggaatcgttcttgacgtgaaaatctttaaccgtgaagacggcgatgaacttcctccaggcgtcaaccaacttgtccgtgtctacattgtacaaaagcggaaaattaaccagggcgataaaatggctggccgtcacgggaacaaaggtgttatttcccgaatcctgccagaagaagatatgccgtttttaccagacggaacgcctgttgacattatgttaaacccacttggcgtgccttcgcggatgaatatcggacaagtgcttgaactccatcttggtatggctgcccgcaagctaggcatccatgttgcgtctccagtatttgacggcgccagtgaagaagatgtttggggcacattggaagaagcaggcatggcccgagacggaaaaacaattttatatgatggccgtactggcgaaccgtttgacaaccgcgtatcagttgggattatgtatatgatcaagcttgcccatatggttgaacgaccaagctccatgcgcgtcaacaggcccgtaactcattcgttaacgcagcagcttcttggcggtaaagcccaatttggtggacagcgtttcggggagatggaagtatgggcactggaagcatatggtgctgcttacacgcttccaagaaatccttcactgttaaattcagatgacgttgttgggcgtgtgaaaacgtacgaggcaattgtaaaaggggaaaacgttcctgagccaggggtgccagaatcgtttaaagtcctcattaaagaattgcaatcgttgggcatggatgtgaagatgctctccagcaacgaagaggaaattgaaatgcgtgagctggatgacgaagaagaccaaacgtctgaaaaactcaacctcaacttagagacgaacgaatcacaacta

SEQ ID NO: 4 - protein, 1182 aa, Bacillus clausii
  misc_feature (140)..(140): The ′Xaa′ at location 140 stands for Leu.
LTGQLIQYGRHRQRRSYARINEVLELPNLIEIQTASYQWFLDEGLREMFQDISPIQDFTGNLVLEFIDYSLGEPKYPVDESKERDVTFAAPLRVKVRLINKETGEVKEQEVFMGDFPLMTETGTFIINGAERVIVSQLVXSPSVYYSQKLDKNGKKGFTATVIPNRGAWLELETDAKDIVYVRIDRTRKIPVTVLLRALGFGSDQEIVDLLGENEYLRNTLEKDNTDSTDKVLLEIYERLRPGEPPTVENAKSLLESRFFDPKRYDLANVGRYKINKKLHIKNRLFNQRLAEKLVDPETGEVLAEEGTLLDRRTLDKLIPHLEKNVGFRTARTSGGVLEESDVEIQSVKIYVADDYEGERVISVISNGMVERDVKHIAPADIIASISYFFNLLHGVGDTDDIDHLGNRRLRSVGELLQNQFRIGLSRMERVVRERMSIQDPNVITPQALINIRPVIASIKEFFGSSQLSQFMDQTNPLAELTHKRRLSALGPGGLTRERAGMEVRDVHYSHYGRMCPIETPEGPNIGLINTLSSYAKVNEFGFMETPYRRVDPETGKVTARIDYLTADEEDNYVVAQANAKLNEDGSFVDDNIIARFRGENTVVPCDRVDYMDVSPKQVVSAATSCIPFLENDDSNRALMGANMQRQAVPLLVPEAPLVGTGMEHVSAKDSGAAVVSKYAGIVERVTAKEIWVRRIEEVDGKETKGDLDKYKLQKFVRSNQGTSYNQRPIVREGDRVEKREILADGPSMEMGEMALGRNVLVAFMTWDGYNYEDAIILSERLVKDDVYTSIHIEEYESDARDTKLGPEEITRDIPNVGKNALRNLDERGIIRIGAEVKDGDILVGKVTPKGVTELTAEERLLHAIFGEKAREVRDTSLRAPHGGDGIVLDVKIFNREDGDELPPGVNQLVRVYIVQKRKINQGDKMAGRHGNKGVISRILPEEDMPFLPDGTPVDIMLNPLGVPSRMNIGQVLELHLGMAARKLGIHVASPVFDGASEEDVWGTLEEAGMARDGKTILYDGRTGEPFDNRVSVGIMYMIKLAHMVERPSSMRVNRPVTHSLTQQLLGGKAQFGGQRFGEMEVWALEAYGAAYTLPRNPSLLNSDDVVGRVKTYEAIVKGENVPEPGVPESFKVLIKELQSLGMDVKMLSSNEEEIEMRELDDEEDQTSEKLNLNLETNESQL

SEQ ID NO: 5 - protein, 40 aa, Bacillus subtilis
FFGSSQLSQFMDQTNPLAELTHKRRLSALGPGGLTRERAG

SEQ ID NO: 6 - protein, 40 aa, Synechocystis sp. strain PCC6803.II
FFGSSQLSQFMDQTNPLAELTHKRRISALGPGGLTRERAG

SEQ ID NO: 7 - protein, 40 aa, Neisseria menigitidis Z2491
FFGSSQLSQFMDQTNPLSEVTHKRRVSALGPGGLTRERAG

SEQ ID NO: 8 - protein, 40 aa, Staphylococcus aureus
FFGSSQLSQFMDQANPLAELTHKRRLSALGPGGLTRERAG

SEQ ID NO: 9 - protein, 40 aa, Escherichia coli
FFGSSQLSQFMDQNNPLSEITHKRRISALGPGGLTRERAG

SEQ ID NO: 10 - protein, 40 aa, Haemophilus influenzae
FFGSSQLSQFMDQNNPLSEVTHKRRISALGPGGLTRERAG

SEQ ID NO: 11 - protein, 40 aa, Chlamydia pneumoniae
FFGRSQLSQFMDQTNPVAELTHKRRLSALGPGGLNRERAG

SEQ ID NO: 12 - protein, 40 aa, Coxiella burnetii
FFGSSQLSQFMDQNNPLSEITHKRRVSALGPGGLTRERAG

SEQ ID NO: 13 - protein, 40 aa, Pseudomonas aeruginosa; identical to SEQ ID NO: 12

SEQ ID NO: 14 - protein, 40 aa, Pseudomonas putida
FFGSSQLSQFMDQNNPLSEITHKRRCSALGPGGLTRERAG

SEQ ID NO: 15 - protein, 40 aa, Samonella typhimurium
FFGSSQLSQFMDQNNPLSEITHKRRISALGPGGLTRELAG

SEQ ID NO: 16 - protein, 40 aa, Mycobacterium leprae
FFGTSQLSQFMDQNNPLSGLTHKRRLSALGPGGLSRERAG

SEQ ID NO: 17 - protein, 40 aa, Borrelia burgdorferi
FFATSQLSQFMDQVNPLAELTHKRRLNALGPGGLSRDRAG

SEQ ID NO: 18 - protein, 40 aa, Spiroplasma citri
FFNLSQLSQFMDQTNPLAELTNKRRLTALGPGGLSRERAG

SEQ ID NO: 19 - protein, 40 aa, Campylobacter jejuni
FFTGGQLSQFMDQTNPLSEVTHKRRLSALGEGGLVKERAG

SEQ ID NO: 20 - protein, 40 aa, Aquifex aeolicus
FLKTGQLSQYLDNTNPLSELTHKRRLSALGPGGLTRESAG

SEQ ID NO: 21 - protein, 40 aa, Thermotoga maritima
FFAMNQLSQFMDQVNPLSELTHKRRVSAVGPGGLRRERAG

SEQ ID NO: 22 - protein, 40 aa, Bacillus sp. strain C-125; identical to SEQ ID NO: 5

SEQ ID NO: 23 - protein, 40 aa, Xylella fastidiosa
FFGSSQLSQFMDQNNPLSEVTHKRRLSALGPGGLTRERAG

SEQ ID NO: 24 - protein, 40 aa, Klebsiella ornithinolytica; identical to SEQ ID NO: 9

SEQ ID NO: 25 - protein, 40 aa, Calymmatobacterium granulomatis; identical to SEQ ID NO: 9

SEQ ID NO: 26 - protein, 40 aa, Serratia marcescens; identical to SEQ ID NO: 9

SEQ ID NO: 27 - protein, 40 aa, Shewanella violacea; identical to SEQ ID NO: 10

SEQ ID NO: 28 - protein, 40 aa, Streptococcus pneumoniae
FFGSSQLSQFMDQHNPLSELSHKRRLSALGPGGLTRDRAG

SEQ ID NO: 29 - protein, 40 aa, Legionella pneumophila
FFGSSQLSQFMDQVNPLSGVTHKRRVSALGPGGLTRERAG

SEQ ID NO: 30 - protein, 40 aa, Deinococcus radiodurans R1
FFGRSQLSQFKDQTNPLSDLRHKRRISALGPGGLTRERAG

SEQ ID NO: 31 - protein, 40 aa, Streptomyces coelicolor
FFGTSQLSQFMDQNNPLSGLTHKRRLNALGPGGLSRERAG

SEQ ID NO: 32 - protein, 40 aa, Helicobacter pylori
FFMGGQLSQFMDQTNPLSEVTHKRRLSALGEGGLVKDRVG

(19) United States
(12) Patent Application Publication
(10) Pub. No.: US 2009/0317865 A1
(43) Pub. Date: Dec. 24, 2009

(54) Eubacterial RNA-Polymerase Mutants With Altered Product Production

(75) Inventors: Steen Troels Jørgensen, Alleroed (DK); Jens Toenne Andersen, Naerum (DK); Niels Banke, Soeborg (DK); Preben Nielsen, Hoersholm (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/545,535

(22) Filed: Aug. 21, 2009

Related U.S. Application Data
(63) Continuation of application No. 10/498,302, filed on Jun. 8, 2004.
(60) Provisional application No. 60/346,675, filed on Jan. 8, 2002.
(60) Provisional application No. 60/359,062, filed on Feb. 21, 2002.

(30) Foreign Application Priority Data
Dec. 29, 2001 (DK) 2001 01972
Feb. 21, 2002 (DK) 2002 00274

(51) Int. Cl. C12P 21/02; C12N 1/21; C12P 1/00

(52) U.S. Cl. 435/69.1; 435/252.31; 435/41

(57) ABSTRACT

The present invention relates to an isolated mutant eubacterium comprising at least one mutation resulting in a substitution of at least one amino acid in the beta-subunit of the RNA-polymerase encoded for by the rpoB-gene providing an altered production of a product of interest when said production of a product of interest is compared to the production of the same product in an isogenic wild type strain grown at identical conditions, wherein the substitution of at least one amino acid occurs at any of positions 469, 478, 482, 485, or 487 of SEQ ID NO:2, or at the equivalent positions in any eubacterial RNA-polymererase beta-subunit family member. Another aspect of the invention relates to a process for producing at least one product of interest in a mutant eubacterium and to a use of the mutant eubacterium according to the invention for producing at least one product of interest.

### CROSS-REFERENCE TO RELATED APPLICATIONS

This application is a continuation of U.S. application Ser. No. 10/498,302 filed Jun. 8, 2004, which is a 35 U.S.C. 371 national application of PCT/DK02/00886 filed Dec. 20, 2002, which claims priority or the benefit under 35 U.S.C. 119 of Danish application nos. PA2001 01972 and PA200200274 filed Dec. 29, 2001 and Feb. 21, 2002, respectively, and U.S. provisional application Nos. 60/346,675 and 60/359,062 filed Jan. 8, 2002 and Feb. 21, 2002, respectively, the contents of which are fully incorporated herein by reference.

### FIELD OF THE INVENTION

The present invention relates to an isolated mutant eubacterium comprising at least one mutation resulting in a substitution of at least one amino acid in the beta-subunit of the RNA-polymerase encoded for by the rpoB-gene providing an altered production of a product of interest when said production of a product of interest is compared to the production of the same product in an isogenic parent strain grown at identical conditions. Another aspect of the invention relates to a process for producing at least one product of interest in a mutant eubacterium and to a use of the mutant eubacterium according to the invention for producing at least one product of interest.

### BACKGROUND OF THE INVENTION

In the industrial production of polypeptides it is of interest to achieve a product yield as high as possible. One way to increase the yield is to increase the copy number of a gene encoding a polypeptide of interest. This can be done by placing the gene on a high copy number plasmid. However, plasmids are unstable and are often lost from the host cells if there is no selective pressure during the cultivation of the host cells. Another way to increase the copy number of the gene of interest is to integrate it into the host cell chromosome in multiple copies. It has previously been described how to integrate a gene into the chromosome by double homologous recombination without using antibiotic markers (Hone et al., 1988, Microbial Pathogenesis 5: 407-418); integration of two genes has also been described (Novo Nordisk: WO 91/09129 and WO 94/14968). Integrating several copies of a gene into the chromosome of a host cell could lead to instability. Integration of two genes closely spaced in anti-parallel tandem to achieve better stability has been described (Novozymes: WO 99/41358) as well as the stable chromosomal multi-copy integration of genes (Novozymes: WO 02/00907).

Other ways of increasing the product yield would be to increase promoter activity of the specific promoter regulating the expression of a specific gene of interest. Also a more general increase in the activity of several promoters at the same time could lead to an improved product yield.

The most studied bacterial RNA-polymerase is the E. coli RNA-polymerase and it is representative of the enzymes isolated from a number of bacterial genera, including Salmonella, Serratia, Proteus, Aerobacter, and Bacillus (Fukuda et al., 1977, Mol. Gen. Genet. 154:135).

The RNA-polymerase core enzyme is composed of alpha, beta, and beta′ subunits in the ratio 2:1:1, encoded for by the rpoA, rpoB, and rpoC genes respectively. Mutations that confer a cellular resistance to several antibiotics (e.g., rifampicin) are within the rpoB gene.

Rifampicin binds to the beta-subunit and completely blocks productive initiation of RNA chains by the enzyme in vitro and in vivo (Wehrli and Staehelin, 1971, Bact. Rev. 35:290). Cells gain resistance to rifampicin by virtue of an altered beta subunit that fails to bind the drug.

Mutations in the beta-subunit of the RNA-polymerase of rifampicin resistant cells map in three separate regions within a 200-amino-acid stretch in the center of the subunit, and these have been mentioned as a putative rifampicin-binding pocket (Jin and Gross, 1988, J. Mol. Biol. 202: 45-58).

Mutations in rpoB resulting in rifampicin resistance have been reported to cause highly pleiotropic phenotypes (Yang and Price, 1995, J. Biol. Chem. 270: 23930-23933; Kane et al., 1979, J. Bacteriol. 137: 1028-1030). In some cases resulting in an increase in gene activity and in other cases with no effect (Kane et al., 1979, J. Bacteriol. 137: 1028-1030). Sharipova et al. (1994, Microbiology 63: 29-32) have reported higher levels of phosphatase activity for some rifampicin resistant strains and lower levels for other rifampicin resistant strains. A 100-fold improved alpha-amylase activity has been reported in a rifampicin resistant Spo⁻ isolate of Bacillus licheniformis (Hu Xuezhi et al., 1991, Acta microbiologica sinica 31: 268-273). In Bacillus subtilis the mutation Q469R results in rifampicin resistance and hypersensitivity to NusG (Ingham and Furnaux, 2000, Microbiology 146: 3041-3049).

### SUMMARY OF THE INVENTION

Though some reports have indicated an increased productivity of some gene products in rifampicin resistant isolates the effects of mutations in rpoB have been reported to be highly pleiotropic and the observed increase in productivity have never been linked to specific mutations in rpoB. Surprisingly it has now been discovered that the specific mutations of the present invention alone or in combination will result in a much improved productivity of a product of interest when the said rpoB-mutation(s) are present in the host strain.

In a first aspect the present invention relates to an isolated mutant eubacterium comprising at least one mutation resulting in a substitution of at least one amino acid in the beta-subunit of the RNA-polymerase encoded for by the rpoB-gene providing an altered production of a product of interest when said production of a product of interest is compared to the production of the same product in an isogenic parent strain grown at identical conditions, wherein the substitution of at least one amino acid occurs at any of positions 469, 478, 482, 485, and 487 in SEQ ID NO: 2, or at the equivalent positions in any eubacterial RNA-polymererase beta-subunit family member.

In a second aspect the present invention relates to a process for producing at least one product of interest in a mutant eubacterium comprising cultivating the mutant eubacterium as defined above in a suitable medium whereby the said product is produced.

In a third aspect the present invention relates to a use of the mutant eubacterium according to the invention for producing at least one product of interest comprising cultivating the mutant eubacterium in a suitable medium whereby the said product is produced.

### BRIEF DESCRIPTION OF DRAWINGS

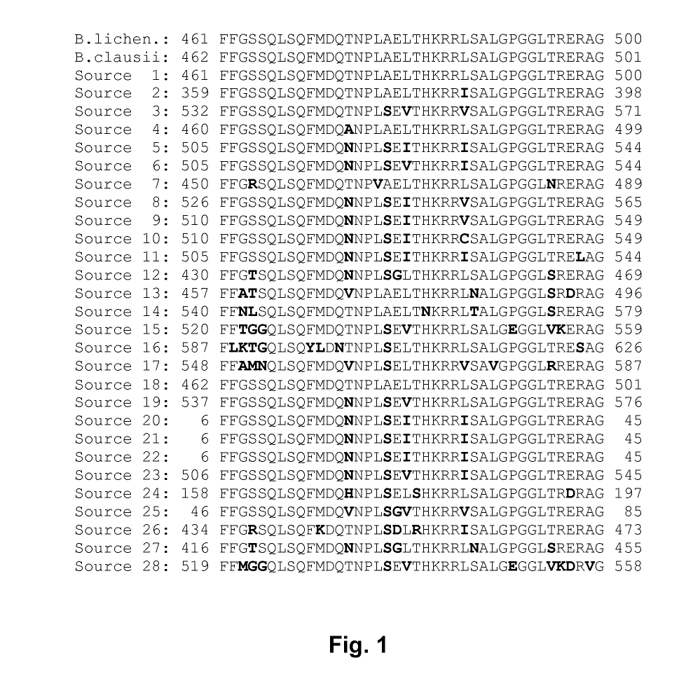

The top line of the aligned sequences in FIG. 1 shows the 40 amino acid segment of the B. licheniformis RpoB protein, from position 461 to 500 both incl. of SEQ ID NO:2. The second line shows the homologous 40 amino acid segment of the Bacillus clausii RpoB protein, from position 462 to 501 both incl. of SEQ ID NO:4, aligned with the corresponding positions of SEQ ID NO:2. These two segments are aligned in FIG. 1 with published sequences of RpoB proteins from different bacteria (SEQ ID NOs: 5-32), letters in bold indicate wildtype deviations from the RpoB sequence of the two Bacillus sequences at the top. The microbial sources of the homologous segments are indicated below for each source number in the figure:

Source 1) Boor et al., Genetic and transcriptional organization of the region encoding the beta subunit of Bacillus subtilis RNA polymerase. J. Biol. Chem. 270:20329 (1995).

Source 2) Kaneko et al., Sequence analysis of the genome of the unicellular cyanobacterium Synechocystis sp. strain PCC6803. II. Sequence determination of the entire genome and assignment of potential protein-coding regions. DNA Res. 3:109 (1996).

Source 3) Parkhill et al., Complete DNA sequence of a serogroup A strain of Neisseria menigitidis Z2491. Nature 404:502 (2000).

Source 4) Aboshkiwa et al., Cloning and physical mapping of the Staphylococcus aureus rplL, rpoB and rpoC genes, encoding ribosomal protein L7/L12 and RNA polymerase subunits beta and beta′. J. Gen. Microbiol. 138:1875 (1992).

Source 5) Ovchinnikov et al., The primary structure of Escherichia coli RNA polymerase. Nucleotide sequence of the rpoB gene and amino-acid sequence of the beta-subunit. Eur. J. Biochem. 116:621 (1981).

Source 6) Fleischmann et al., Whole-genome random sequencing and assembly of Haemophilus influenzae Rd. Science 269:496 (1995).

Source 7) Kalman et al., Comparative genomes of Chlamydia pneumoniae and C. trachomatis. Nat. Genet. 21:385 (1999).

Source 8) Mollet et al., Determination of Coxiella burnetii rpoB sequence and its use for phylogenetic analysis. Gene 207:97 (1998)

Source 9) Stover et al., Complete genome sequence of Pseudomonas aeruginosa PAO1, an opportunistic pathogen. Nature 406:959 (2000)

Source 10) Borodin et al., Nucleotide sequence of the rpoB gene coding for the beta-subunit of RNA polymerase in Pseudomonas putida. Dokl. Biochem. 302:1261 (1988)

Source 11) Sverdlov et al., Nucleotide sequence of the rpoB gene of Samonella typhimurium coding for the beta-subunit of RNA polymerase. Dokl. Biochem. 287:62 (1986)

Source 12) Honore et al., Nucleotide sequence of the first cosmid from the Mycobacterium leprae genome project: structure and function of the Rif-Str regions. Mol. Microbiol. 7:207 (1993).

Source 13) Alekshun et al., Molecular cloning and characterization of Borrelia burgdorferi rpoB. Gene 186:227 (1997).

Source 14) Laigret et al., The unique organization of the rpoB region of Spiroplasma citri: a restriction and modification system gene is adjacent to rpoB. Gene 171:95 (1996).

Source 15) Parkhill et al., The genome sequence of the food-borne pathogen Campylobacter jejuni reveals hypervariable sequences. Nature 403:665 (2000).

Source 16) Deckert et al., The complete genome of the hyperthermophilic bacterium Aquifex aeolicus. Nature 392:353 (1998)

Source 17) Palm et al., The DNA-dependent RNA-polymerase of Thermotoga maritima; characterisation of the enzyme and the DNA-sequence of the genes for the large subunits. Nucleic Acids Res. 21:4904 (1993).

Source 18) Takaki et al., Sequence analysis of a 32-kb region including the major ribosomal protein gene clusters from alkaliphilic Bacillus sp. strain C-125. Biosci. Biotechnol. Biochem. 63:452 (1999).

Source 19) Simpson et al., The genome sequence of the plant pathogen Xylella fastidiosa. Nature 406:151 (2000).

Source 20) Drancourt, M. Klebsiella ornithinolytica, Klebsiella taxonomy. Submitted (FEB-1999) to the EMBL/GenBank/DDBJ databases.

Source 21) Drancourt and Raoult, Calymmatobacterium granulomatis rpoB. Submitted (DEC-1999) to the EMBL/GenBank/DDBJ databases.

Source 22) Mollet et al., (Serratia marcescens) RNA polymerase beta-subunit. Submitted (NOV-1996) to the EMBL/GenBank/DDBJ databases.

Source 23) Nakasone et al., Isolation of rpoB and rpoC genes from deep-sea piezophilic bacterium Shewanella violacea and its overexpression in Escherichia coli. Submitted (JUL-2000) to the EMBL/GenBank/DDBJ databases.

Source 24) Padayachee and Klugman, Molecular basis for rifampicin resistant Streptococcus pneumoniae isolates from South Africa. Submitted (FEB-1999) to the EMBL/GenBank/DDBJ databases.

Source 25) Nielsen et al., Legionella pneumophila RNA polymerase B-subunit (rpoB) gene. Submitted (DEC-1998) to the EMBL/GenBank/DDBJ databases

Source 26) White et al., Genome sequence of the radioresistant bacterium Deinococcus radiodurans R1. Science 286:1571 (1999).

Source 27) Streptomyces coelicolor, Seeger and Harris, Submitted (March-2000) to the EMBL/GenBank/DDBJ databases.

Source 28) Helicobacter pylori (Campylobacter pylori), Hocking et al., Submitted (January-1997) to the EMBL/GenBank/DDBJ databases.

### DEFINITIONS

Prior to a discussion of the detailed embodiments of the invention, a definition of specific terms related to the main aspects of the invention is provided. In the present description and claims, the conventional one-letter codes for nucleotides and the conventional one-letter and three-letter codes for amino acid residues are used. For ease of reference the following nomenclature is used:
Original amino acid(s) position(s) substituted amino acid(s)

According to this nomenclature, and by way of example, the substitution of asparagine for alanine in position 30 is shown as:
Ala 30 Asn or A30N
   a deletion of alanine in the same position is shown as:
Ala 30* or A30*
   and insertion of an additional amino acid residue, such as lysine, is shown as:
Ala 30 AlaLys or A30AK

A deletion of a consecutive stretch of amino acid residues, exemplified by amino acid residues 30-33, is indicated as (30-33)*.

Where a specific polypeptide contains a deletion (i.e., lacks an amino acid residue) in comparison with homologous polypeptides, and an insertion is made in such a position, this is indicated as:
*36 Asn or*36N
   for insertion of an asparagine in position 36.

Multiple mutations are separated by plus signs, i.e.:
Ala 30 Asn+Glu 34 Ser or A30N+E34S
   representing substitutions in positions 30 and 34 (in which asparagine and serine is substituted for alanine and glutamic acid, respectively).

When one or more alternative amino acid residues may be inserted in a given position this is indicated as:
A30N,E or A30N or A30E

Furthermore, when a position suitable for modification is identified herein without any specific modification being suggested, it is to be understood that any other amino acid residue may be substituted for the amino acid residue present in that position (i.e., any amino acid residue—other than that normally present in the position in question—chosen among A, R, N, D, C, Q, E, G, H, I, L, K, M, F, P, S, T, W, Y and V). Thus, for instance, when a modification (replacement) of a methionine in position 202 is mentioned, M202, but not specified, it is to be understood that any of the other amino acids may be substituted for the methionine, i.e., any other amino acid chosen among A, R, N, D, C, Q, E, G, H, I, L, K, F, P, S, T, W, Y and V.

Eubacterium: The term “eubacterium” in the context of the present invention means unicellular prokaryotic microorganisms possessing cell walls, with cells in the form of rods, cocci or spirilla, many species motile with cells bearing one or more flagella. Eubacteria are distinguished from archaebacteria by the possession of peptidoglycan cell walls and ester-linked lipids.

Mutant eubacterium: The term “mutant eubacterium” in the context of the present invention means the otherwise isogenic parent eubacterium which has obtained a mutation in at least one of the five claimed positions in the beta-subunit of the RNA-polymerase.

Substitution: The term “substitution” in the context of the present invention means the replacement of one amino acid with another amino acid.

Beta-subunit: The term “beta-subunit” in the context of the present invention means the RpoB-protein encoded for by the rpoB gene, and which subunit is comprised in the RNA-polymerase composed of alpha, beta, beta′ subunits in the ratio 2:1:1 and encode for by the genes rpoA, rpoB, and rpoC respectively.

rpoB-gene: The term “rpoB-gene” in the context of the present invention means the gene encoding the beta-subunit of the RNA-polymerase.

Beta-subunit family member: The term “beta-subunit family member” in the context of the present invention does not mean family in the normal taxonomic sense where a family comprises members of the same genus, rather in the present context the term refers to any prokaryotic (eubacterial) RNA-polymerase composed of three subunits alpha, beta, and beta′ in the above mentioned ratio and wherein the amino acid sequence of said beta-subunit comprises a 40 amino acid contiguous sequence that can be aligned with the sequence of the RpoB protein from position 461 to 500 of SEQ ID NO:2 and result in at least 75% homology.

Isogenic parent strain: The term “isogenic parent strain” in the context of the present invention means a strain which is genetically identical to the progeny mutant or mutant strain of the present invention, except for the at least one mutation in the rpoB-gene of the said mutant.

Equivalent positions: The term “equivalent positions” in the context of the present invention means the positions after alignment with the segment from position 461 to 500 of SEQ ID NO:2 as also illustrated in FIG. 1 and in example 3.

Homology: The term “homology” in the context of the present invention relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wis., USA 53711) (Needleman and Wunsch, 1970, Journal of Molecular Biology 48: 443-453). Using GAP with the following settings for DNA sequence comparison: GAP creation penalty of 5.0 and GAP extension penalty of 0.3.”

Metabolic pathway: The term “metabolic pathway” in the context of the present invention means a chain of enzyme-catalyzed biochemical reactions in living cells which, e.g., convert one compound into another, or build up large macromolecules from smaller units, or break down compounds to release usable energy.

Exogenous: The term “exogenous” in the context of the present invention means that e.g., the gene is not normally present in the host organism in nature.

Endogenous: The term “endogenous” in the context of the present invention means that e.g., the gene originates from within the host organism.

Operon: The term “operon” in the context of the present invention means a polynucleotide comprising several genes that are clustered and perhaps even transcribed together into a polycistronic mRNA, e.g., genes coding for the enzymes of a metabolic pathway. The transcription of an operon may be initiated at a promoter region and controlled by a neighboring regulatory gene, which encodes a regulatory protein, which in turn binds to the operator sequence in the operon to respectively inhibit or enhance the transcription.

Altered product production: The term “altered product production” in the context of the present invention means that either the product yield is altered, which means the final amount of product produced per added amount of substrate is altered, or that the same amount of product is obtained by a shorter or longer culture period. An altered product production may be an increased product yield, or an increased productivity, however it may also be of interest to lower product yield, or to lower the productivity of certain products.

Nucleic acid construct: When used herein, the term “nucleic acid construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” is defined herein as a configuration in which a control sequence is appropriately placed at a position relative to the coding sequence of the DNA sequence such that the control sequence directs the expression of a polypeptide.

Coding sequence: When used herein the term “coding sequence” is intended to cover a nucleotide sequence, which directly specifies the amino acid sequence of its protein product. The boundaries of the coding sequence are generally determined by an open reading frame, which usually begins with the ATG start codon. The coding sequence typically include DNA, cDNA, and recombinant nucleotide sequences.

Expression: In the present context, the term “expression” includes any step involved in the production of the polypeptide including, but not limited to, transcription, post-transcriptional modification, translation, post-translational modification, and secretion.

Expression vector: In the present context, the term “expression vector” covers a DNA molecule, linear or circular, that comprises a segment encoding a polypeptide of the invention, and which is operably linked to additional segments that provide for its transcription.

Host cell: The term “host cell”, as used herein, includes any cell type which is susceptible to transformation with a nucleic acid construct.

### DETAILED DESCRIPTION OF THE INVENTION

In E. coli mutations resulting in rifampicin resistance displays pleiotropic effects as discussed above. In E. coli this resistance is due to point mutations in the rpoB gene (Jin and Gross, 1988, J. Mol. Biol. 202: 45-58) mapping within three separate regions of the protein. In some reported cases the mutations also result in changed levels of specific proteins. Among the known rpoB genes from different bacteria that have been sequenced a high degree of homology have been found.

In Bacillus licheniformis we have determined the amino acid sequence of the RpoB protein to be about 95% homologous to the RpoB protein from Bacillus subtilis. Four co-linear blocks of sequence similarity are shared between the B. subtilis and E. coli beta-subunits including conserved regions where alterations causing rifampicin resistance maps. In the present invention rpoB-mutants of B. licheniformis were isolated and tested for the production of a product of interest. RpoB-mutants can be obtained by known techniques in the art such as site directed mutagenesis or random mutagenesis. Also several positions in the RpoB-protein has previously been shown when mutated to result in rifampicin resistance.

In the present invention rifampicin resistant RpoB mutants of B. licheniformis and B. clausii were obtained, and mutants carrying different individual mutations in RpoB were tested for altered product production of a product of interest.

The analysis of the isolated mutants showed, that rifampicin resistance in B. licheniformis and B. clausii is due to point mutations in the rpoB gene, and furthermore specific mutations were identified, that consistently resulted in an increase in the production of specific products of interest as shown in examples 4 and 5.

In most cases a single substitution of one amino acid in one of 5 specific positions in the RpoB protein will result in an improved production of a product of interest, there may be synergistic effects of combining two or more of the specific mutations.

In a first aspect the present invention therefore relates to an isolated mutant eubacterium comprising at least one mutation resulting in a substitution of at least one amino acid in the beta-subunit of the RNA-polymerase encoded for by the rpoB-gene providing an altered production of a product of interest when said production of a product of interest is compared to the production of the same product in an isogenic parent strain grown at identical conditions, wherein the substitution of at least one amino acid occurs at any of positions 469, 478, 482, 485, or 487 in SEQ ID NO:2, or at the equivalent positions in any eubacterial RNA-polymererase beta-subunit family member.

In another embodiment the altered product production is an improved production.

From the DNA sequence of the isolated mutants several specific mutations resulting in amino acid substitutions of at least one amino acid and providing an improved production of a product of interest have been identified. In one embodiment the present invention therefore relates to a substitution of at least one amino acid in the in the beta-subunit of the RNA-polymerase encoded for by the rpoB-gene as described above, wherein the said at least one substitution of at least one amino acid comprises Q469R, A478D, A478V, H482R, H482P, R485H, or S487L, wherein the positions correspond to the equivalent positions of those beta-subunits when aligned with SEQ ID NO:2.

Once the relevant positions resulting in an improved product production has been identified it will be obvious to the skilled person to try random substitutions at the same positions and in a further embodiment the present invention relates to an isolated mutant eubacterium of the first aspect wherein the said substitution of at least one amino acid comprises any random substitution at position 469 or 478 in SEQ ID NO:2, or wherein the substitution of at least one amino acid comprises any substitution at positions 469, 478, and/or 487 in SEQ ID NO:2, or at the equivalent position(s) in any eubacterial RNA-polymerase beta-subunit family member.

In a particular embodiment the present invention relates to an isolated mutant eubacterium as defined above, wherein the substitution of at least one amino acid comprises Q469R, A478D, and/or S487L; or preferably wherein the said at least one substitution of at least one amino acid comprises Q469R or A478D; wherein the positions correspond to the equivalent positions of those beta-subunits when aligned with SEQ ID NO:2.

The isolated bacterium according to the present invention should comprise a RNA-polymerase composed of three subunits alpha, beta, and beta′ in the above mentioned ratio and wherein the amino acid sequence of said beta-subunit comprises a 40 amino acid contiguous sequence that can be aligned with the sequence of the RpoB protein from position 461 to 500 of SEQ ID NO:2 and result in at least 75% homology.

In a particular embodiment the said homology is at least 80%. In a further particular embodiment the homology is 85% and in a still further embodiment the homology is 90%.

In some bacterial genera, e.g., some gram-negative bacteria like Escherichia, Salmonella, Neiseria and Pseudomonas, the equivalent position to position 478 in B. licheniformis (SEQ ID NO:2) is normally serine (S) instead of alanine (A), as evident from FIG. 1, in which the amino acid segment from position 461 to 500 in SEQ ID NO:2 from B. licheniformis has been aligned with published sequences of RpoB-proteins from various bacteria.

The amino acids, serine and alanine, are very similar in structure and it is therefore not surprising that it will be possible to replace alanine with serine or the other way around, without affecting the functionality of the RpoB-protein.

The claimed substitution of an amino acid at position 478 from e.g., A478D or A478V, or a random substitution of A478 should therefore in the context of the present invention also comprise S478D or S478V, or any random substitution of S478.

In the first aspect of the present invention the bacterium is an isolated mutant eubacterium. In one embodiment the isolated mutant eubacterium is a gram positive bacterium. Useful gram positive bacteria include but are not limited to Bacillus sp., e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis.

In another embodiment the said bacterium comprises Bacillus lentus, Bacillus licheniformis, Bacillus amyloliquefaciens, Bacillus clausii, Bacillus stearothermophilus, and Bacillus subtilis.

In a further embodiment of the present invention the said bacterium is a Bacillus licheniformis.

According to the present invention the product of interest comprises a gene product or a product of a metabolic pathway the production of which is improved as a result of the at least one substitution of at least one amino acid in the in the beta-subunit of the RNA-polymerase encoded for by the rpoB-gene.

One preferred embodiment of the invention relates to a mutant of the first aspect, wherein the product of interest is a product of a gene or a metabolic pathway; preferably the gene encoding the product of interest is comprised in an operon; even more preferably the gene is exogenous or endogenous to the mutant eubacterium; still more preferably the gene is present in at least two copies or in at least ten copies or even in at least 100 copies.

The gene or the operon can be carried on a suitable plasmid that can be stably maintained, e.g., capable of stable autonomous replication in the host cell (the choice of plasmid will typically depend on the compatibility of the plasmid with the host cell into which the plasmid is to be introduced) or it can be carried on the chromosome of the host. The said gene may be endogenous to the host cell in which case the product of interest is a protein naturally produced by the host cell and in most cases the gene will be in it normal position on the chromosome. If the gene encoding the product of interest is an exogenous gene, the gene could either be carried on a suitable plasmid or it could be integrated on the host chromosome. In one embodiment of the invention the eubacterium is a recombinant eubacterium. Also the product of interest may in another embodiment be a recombinant protein.

Integration of the gene encoding the product of interest may be achieved in several ways known to the skilled person. The gene may be present in one, two or more copies on the chromosome.

Integration of two genes has been described in WO 91/09129 and WO 94/14968 (Novo Nordisk) the content of which is hereby incorporated by reference. Integration of two genes closely spaced in anti-parallel tandem to achieve better stability has been described in WO 99/41358 (Novo Nordisk) the content of which is hereby incorporated by reference, as well as the stable chromosomal multi-copy integration of genes described in WO 02/00907 (Novozymes A/S) the content of which is incorporated herein by reference.

The presence of the gene encoding the product of interest in several copies may also further improve the production of the said product. If integrated on the chromosome of the host cell the gene may be present in one, two, or more copies per chromosome. If carried on a plasmid the gene may be present in several hundred copies per cell. In one embodiment of the present invention the said gene is present in at least two copies. In another embodiment the said gene is present in at least ten copies, and in a still further embodiment of the present invention the gene is present in at least 100 copies.

Selection of chromosomal integrant has for convenience resulted in the use of selectable markers such as antibiotic resistance markers. However it is desirable if possible to avoid the use of antibiotic marker genes. WO 01/90393 discloses a method for the integration of a gene in the chromosome of a host cell without leaving antibiotic resistance markers behind in the strain, the content of which is hereby incorporated by reference.

In a further embodiment the present invention thus relates to an isolated mutant eubacterium as defined above, wherein the gene encoding the said product of interest is integrated on the mutant eubacterial host chromosome without leaving any antibiotic resistance marker genes at the site of integration.

The present invention also relates to nucleic acid constructs comprising a nucleotide sequence encoding a product of interest, which may be operably linked to one or more control sequences that direct the expression of the coding sequence in a suitable host cell under conditions compatible with the control sequences.

A nucleotide sequence encoding a polypeptide of interest may be manipulated in a variety of ways to provide for expression of the polypeptide. Manipulation of the nucleotide sequence prior to its insertion into a vector may be desirable or necessary depending on the expression vector. The techniques for modifying nucleotide sequences utilizing recombinant DNA methods are well known in the art.

The control sequence may be an appropriate promoter sequence, a nucleotide sequence which is recognized by a host cell for expression of the nucleotide sequence. The promoter sequence contains transcriptional control sequences, which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (VIIIa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

### Expression Vectors

The present invention also relates to recombinant expression vectors comprising the nucleic acid construct of the invention. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers which confer antibiotic resistance such as ampicillin, kanamycin, chloramphenicol or tetracycline resistance.

The vectors of the present invention preferably contain an element(s) that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. The origin of replication may be one having a mutation which makes its functioning temperature-sensitive in the host cell (see, e.g., Ehrlich, 1978, Proceedings of the National Academy of Sciences USA 75: 1433).

More than one copy of a nucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant a host cell comprising the nucleic acid construct of the invention, which are advantageously used in the recombinant production of the polypeptides. A vector comprising a nucleotide sequence of the present invention is introduced into a host cell so that the vector is maintained as a chromosomal integrant or as a self-replicating extra-chromosomal vector as described earlier. The host cell may be a unicellular microorganism, e.g., a prokaryote, or a non-unicellular microorganism, e.g., a eukaryote.

Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The product of interest is any gene product or product of a metabolic pathway which is industrially useful and which can be produced in a bacterial cell such as an eubacterium. In one embodiment the product of interest comprises polypeptides, vitamins, amino acids, antibiotics, carbohydrates, or surfactants.

A preferred embodiment relates to a mutant of the first aspect, wherein the product of interest comprises polypeptides, vitamins, amino acids, antibiotics, carbohydrates, or surfactants; preferably the product of interest is a polypeptide, preferably an enzyme; still more preferably enzyme is an enzyme of a class selected from the group of enzyme classes consisting of oxidoreductases (EC 1), transferases (EC 2), hydrolases (EC 3), lyases (EC 4), isomerases (EC 5), and ligases (EC 6).

In another embodiment the enzyme is an enzyme with an activity selected from the group of enzyme activities consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase. Preferably the enzyme is an amylase or a mannanase.

In yet another embodiment the product is a polypeptide comprising cellulose binding domains, starch binding domains, antibodies, antimicrobial peptides, hormones, or fusion polypeptides. It is preferred, that the product of interest is a polypeptide which comprises cellulose binding domains, starch binding domains, antibodies, antimicrobial peptides, hormones, or fusion polypeptides. It is also preferred, that the product of interest is a carbohydrate, preferably hyaluronic acid.

When present in the host cell the specific mutations of the present invention result in an altered production of a product of interest. The altered production could e.g., be an improved yield or an improved productivity as defined above.

The altered production according to the invention is in one embodiment at least an additional 5% to 1000% of the normal level in the isogenic parent strain grown at identical conditions. In another embodiment the altered production is between 5% to 500%, in a further embodiment between 5% to 250%, and in a still further embodiment between 5% to 100%, and in still another embodiment between 5% to 50%.

The host cells of the present invention are cultured in a suitable nutrient medium under conditions permitting the production of the desired polypeptide, after which the resulting polypeptide optionally is recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g., in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

If the polypeptide is secreted into the nutrient medium, the polypeptide can be recovered directly from the medium. If the polypeptide is not secreted, it can be recovered from cell lysates. The polypeptide may be recovered from the culture medium by conventional procedures including separating the host cells from the medium by centrifugation or filtration, precipitating the proteinaceous components of the supernatant or filtrate by means of a salt, e.g., ammonium sulphate, purification by a variety of chromatographic procedures, e.g., ion exchange chromatography, gelfiltration chromatography, affinity chromatography, or the like, dependent on the type of polypeptide in question.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989).

In one embodiment of the present invention the product of interest is secreted into the nutrient medium. In another embodiment the product is associated with the cell membrane, and in a still further embodiment the product is intracellular.

A second aspect of the invention relates to a process for producing at least one product of interest in a mutant eubacterium comprising cultivating the mutant eubacterium as defined in any of the embodiments of the first aspect of the invention in a suitable medium whereby the said product is produced. Suitable media for the cultivation is described above as well as methods for the purification or isolation of the produced product which is an optional additional step to the process of the present invention.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

A third aspect of the present invention relates to the use of the mutant eubacterium as defined in any of the embodiments of the first aspect of the invention for producing at least one product of interest comprising cultivating the said mutant eubacterium in a suitable medium whereby the said product is produced, and optionally isolating or purifying the produced product.

The present invention is further illustrated by the following examples, which, however, are not to be construed as limiting the scope of protection. The features disclosed in the foregoing description and in the following examples may, both separately and in any combination thereof, be material for realising the invention in diverse forms thereof.

### EXAMPLES

### Example 1

### The DNA Sequence of the rpoB Gene from Bacillus licheniformis

The DNA sequence of the rpoB gene from Bacillus licheniformis was determined from plasmid clones containing fragments of B. licheniformis chromosomal DNA by standard techniques. The plasmid library containing the B. licheniformis clones was obtained from deposit ATCC14580. The DNA sequence is shown in SEQ ID NO:1.

### Example 2

### The B. licheniformis RpoB Protein

The B. licheniformis RpoB protein as translated from the open reading frame of the DNA sequence in SEQ ID NO:1 is shown in SEQ ID NO:2.

### Example 3

### DNA Polymerase Segment Alignment

The 40 amino acid segment of the B. licheniformis RpoB protein from position 461 to 500 in SEQ ID NO:2 was used in a BLAST search against published sequences of RpoB proteins from different bacteria. The alignment is shown in FIG. 1 with the B. licheniformis RpoB sequence at the top.

### Example 4

### Yield/Productivity Improvements in Specific rpoB Mutants

### Materials and Methods

Expression cassettes were integrated in one or more copies into the chromosome of the strains below, as described in WO 91/09129 and WO 99/41358.

### Strains:

JA 677: A B. licheniformis overproducing a variant of its endogenous alpha-amylase (BLA).
JA 678: JA 677 with a mutation in rpoB resulting in the amino acid change Q469R.
JA 688: A B. licheniformis overproducing a variant of the B. stearothermophilus alpha-amylase (BSG).
JA 689: JA 688 with a mutation in rpoB resulting in the amino acid change Q469R.
JA 690: JA 688 with a mutation in rpoB resulting in the amino acid change H482R.
JA 684: A B. licheniformis overproducing the B. amyloliquefaciens alpha-amylase (BAN).
JA 687: JA 684 with a mutation in rpoB resulting in the amino acid change A478D
SJ 4671: A B. licheniformis overproducing its endogenous alpha-amylase (BLA).
SJ 4671 rif10: SJ 4671 with a mutation in rpoB resulting in the amino acid change A478V.
SJ 4490: A B. licheniformis overproducing its endogenous alpha-amylase.
JA 675: SJ4490 with a mutation in rpoB resulting in the amino acid changes Q469R and R485H.
SJ 6129: A B. licheniformis overproducing a variant of a Bacillus alpha-amylase (disclosed in WO 00/60060); (correction—the actual internal ref. no. is SJ 6093).
SJ 6129 rif10: SJ 6129 (SJ 6093) with a mutation in rpoB resulting in the amino acid change S487L.

### Media:

LB agar: 10 g/l peptone from casein; 5 g/l yeast extract; 10 g/l Sodium Chloride; 12 g/l Bacto-agar adjusted to pH 6.8-7.2. LB agar with 50 KAN: LB-agar with 50 mg/l of Kanamycin. M-9 buffer (deionized water is used): Di-Sodiumhydrogenphosphate 2H₂O 8.8 g/l; Potassiumdihydrogenphosphate 3 g/l; Sodium Chloride 4 g/l; Magnesium sulphate 7H₂O 0.2 g/l.
Med-F shake flask media (the concentrations given are after final mixing with deionized water): Part A: Maltodextrin 11 g/l; Casitone 6.2 g/l; Bacto-peptone 0.5; Yeast Extract 0.5 g/l; Magnesium sulphate 7H₂O 0.5 g/l; Calcium chloride 0.1 g/l; Citric acid 50 mg/l; trace metals (MnSO₄H₂O 2.5 mg/l; FeSO₄ 7H₂O 9.9 mg/l; CuSO₄ 5H₂O 1.0 mg/l; ZnCl₂ 1.0 mg/l); Pluronic 0.1 g/l; pH adjusted to 6.7; Part B: 5 g/l Potassiumdihydrogenphosphate pH adjusted to 6.7 with NaOH. After sterilization for 20 min at 121° C. part A and B are mixed 1:1.

### Procedure for Shake Flask Evaluation of Strains:

First the strain was grown on agar slants 1 day at 37° C. (LB-agar for SJ4671; SJ4671rif10; SJ4490; JA675, LB with 50 KAN for JA 677; JA 678; JA 688; JA 689; JA 690; JA684; JA687). The agar was then washed with M-9 buffer, and the optical density at 650 nm (OD_{650 nm}) of the resulting cell suspension was measured.

Each shake flask is inoculated with the same amount of cells based on the OD₆₅₀ₙₘ measurement. An inoculum strength of OD×ml cell suspension=0.1 was used in this case. Each strain was inoculated in 3 shake flasks. The shake flasks were incubated at 37° C. at 300 rpm, and samples were taken after 1, 2 and 3 days. The pH, OD₆₅₀ₙₘ, and α-amylase activity was measured and the relative activity was determined per strain, and in turn the relative activity was calculated to the parent strain for each rpoB mutant strain. Results are shown in Table 1. It is clear from these results that these mutations in rpoB have a significant effect on the productivity/yield of the enzyme.

TABLE 1
Relative amylase activity of rpoB mutant strains (% of parent strain).

| | Day | Day | Day |
|---|---|---|---|
| | 1 | 2 | 3 |
| BLA amylase variant JA677 vs JA678 (Q469R) | 142 | 161 | 168 |
| BSG amylase variant JA688 vs JA689 (Q469R) | 67 | 114 | 107 |
| BSG amylase variant JA688 vs JA690 (H482R) | 195 | 256 | 197 |
| BAN amylase JA684 vs JA687 (A478D) | 162 | 154 | 151 |
| BLA amylase SJ4671 vs SJ4671rif10 (A478V) | 121 | 114 | 112 |
| BLA SJ4490 vs JA675 (Q469R + R485H) | N/A | N/A | 135 |
| Variant amylase SJ 6093 vs SJ 6129 (S487L) | 149 | 122 | 114 |

### Example 5

### The DNA Sequence of a Part of the rpoB Gene from Bacillus clausii

The DNA sequence of a part of the rpoB gene from Bacillus clausii was determined from plasmid clones containing fragments of B. clausii chromosomal DNA by standard techniques. The plasmid library containing the B. clausii clones was obtained from deposit NCIB 10309. The part of the DNA sequence is shown in SEQ ID NO:3.

### Example 6

### The B. clausii RpoB Protein

The B. clausii RpoB protein as translated from the open reading frame of the DNA sequence in SEQ ID NO:3 is shown in SEQ ID NO:4.

### Example 7

### DNA Polymerase Segment Alignment

The 40 amino acid segment of the B. clausii RpoB position 462 to 501 of SEQ ID NO:4, homologous and equivalent in positions to the RpoB from B. licheniformis shown in positions 461 to 500 in SEQ ID NO:2 was used in a BLAST search against published sequences of RpoB proteins from different bacteria. The alignment is shown in FIG. 1 with the B. licheniformis RpoB sequence at the top and B. clausii as the second from the top sequence.

### Example 8

### Yield/Productivity Improvements in Specific rpoB Mutants

### Materials and Methods

Expression was performed from the native expression system.

### Strains:

NCIB 10309: A B. clausii (producing its endogenous protease, BCP).
PP143: A classical mutant of B. clausii NCIB 10309, which has no mutation in DNA-region encoding the 40 amino acid segment of the B. clausii RpoB shown in position 462 to 501 in SEQ ID NO:4 which is homologous to the RpoB of B. licheniformis shown in positions 461 to 500 in SEQ ID NO:2.
NN49201: PP143 with a mutation in the rpoB-gene resulting in the substitution A479D in the encoded RpoB within the 40 amino acid segment shown in position 462 to 501 in SEQ ID NO:4. The substitution A479D in SEQ ID NO:4 corresponds to the equivalent substitution A478D in SEQ ID NO:2, as can clearly be seen from the two top-most lines of the alignment in FIG. 1.

### Media:

Agar: A suitable agar to support good growth of B. clausii e.g., B3-agar: Peptone 6 g/l; Pepticase 4 g/l; Yeast extract 3 g/l; Meat extract 1.5 g/l; Glucose.1H₂O 1 g/l; Agar 20 g/l use of deionised water. pH adjustment to 7.35 with NaOH/HCl; Sterilised at 121° C. for 40 min. After cooling to 40-50° C., 10% v/v of 1 M NaHCO₃, pH 9, sterilized by filtration and 10% v/v of 10% w/v dried skim milk in deionised water, sterilised at 121° C. for 40 min, is added.
M-9 buffer (deionized water is used): Di-Sodiumhydrogenphosphate 2H₂O 8.8 g/l; Potassiumdihydrogenphosphate 3 g/l; Sodium Chloride 4 g/l; Magnesium sulphate 7H₂O 0.2 g/l.
Med-F shake flask media (the concentrations given are after final mixing with deionized water): Part A: Maltodextrin 11 g/l; Casitone 6.2 g/l; Bacto-peptone 0.5; Yeast Extract 0.5 g/l; Magnesium sulphate 7H₂O 0.5 g/l; Calcium chloride 0.1 g/l; Citric acid 50 mg/l; trace metals (MnSO₄H₂O 2.5 mg/l; FeSO₄ 7H₂O 9.9 mg/l; CuSO₄ 5H₂O 1.0 mg/l; ZnCl₂ 1.0 mg/l); Pluronic 0.1 g/l; pH adjusted to 6.7; Sterilization for 20 min at 121° C. Part B: 5 g/l Potassiumdihydrogenphosphate; 28.6 g/l Sodiumcarbonate; 8.4 g/l sodium-hydrogencarbonate pH adjusted to 9.0 with H₃PO₄. This solution is sterile-filtered and used right away. After sterilization part A and B are mixed.

### Procedure for Shake Flask Evaluation of Strains:

First the strain was grown on agar slants 1 day at 37° C. The agar was then washed with M-9 buffer, and the optical density at 650 nm (OD_{650 nm}) of the resulting cell suspension was measured.

Each shake flask is inoculated with the same amount of cells based on the OD₆₅₀ₙₘ measurement. An inoculum strength of OD×ml cell suspension=0.1 was used in this case. Each strain was inoculated in 2 shake flasks. The shake flasks were incubated at 37° C. at 300 rpm, and samples were taken after 1, 2 and 3 days. The pH, OD₆₅₀ₙₘ, and protease activity was measured and the relative activity was determined per strain, and in turn the relative activity was calculated to the parent strain for the rpoB mutant strain. Results are shown in Table 2. It is clear from these results that the mutation in rpoB has a significant effect on the productivity/yield of the enzyme.

TABLE 2
Relative protease activity of rpoB mutant strains (% of parent strain).

| | Day 1 | Day 2 | Day 3 |
|---|---|---|---|
| BCP protease variant PP143 vs | 159 | 144 | 159 |
| NN49201 (A479D) | | | |

1-39. (canceled)

40. An isolated Bacillus licheniformis cell comprising a nucleic acid sequence encoding an RNA-polymerase, wherein
(a) the RNA-polymerase comprises a beta-subunit which comprises
(i) an amino acid sequence which is at least 90% homologous to the amino acid sequence from position 461 to 500 in SEQ ID NO: 2; and
(ii) an amino acid substitution at a position corresponding to position 478 in SEQ ID NO: 2; and
(b) the RNA-polymerase has RNA-polymerase activity.

41. The Bacillus licheniformis cell of claim 40, wherein the beta-subunit comprises
(i) an amino acid sequence which is at least 95% homologous to the amino acid sequence from position 461 to 500 in SEQ ID NO: 2; and
(ii) an amino acid substitution at a position corresponding to position 478 in SEQ ID NO: 2.

42. The Bacillus licheniformis cell of claim 40, wherein the beta-subunit comprises
(i) an amino acid sequence which is at least 98% homologous to the amino acid sequence from position 461 to 500 in SEQ ID NO: 2; and
(ii) an amino acid substitution at a position corresponding to position 478 in SEQ ID NO: 2.

43. The Bacillus licheniformis cell of claim 40, wherein the beta-subunit comprises
(i) the amino acid sequence from position 461 to 500 in SEQ ID NO: 2; and
(ii) an amino acid substitution at a position corresponding to position 478 in SEQ ID NO: 2.

44. The Bacillus licheniformis cell of claim 40, wherein the beta-subunit comprises
(i) the amino acid sequence of SEQ ID NO: 2; and
(ii) an amino acid substitution at a position corresponding to position 478 in SEQ ID NO: 2.

45. The Bacillus licheniformis cell of claim 40, wherein the amino acid substitution is with an aspartic acid residue.

46. The Bacillus licheniformis cell of claim 40, wherein the amino acid substitution is with a valine residue.

47. The Bacillus licheniformis cell of claim 41, wherein the amino acid substitution is with an aspartic acid residue.

48. The Bacillus licheniformis cell of claim 41, wherein the amino acid substitution is with a valine residue.

49. The Bacillus licheniformis cell of claim 42, wherein the amino acid substitution is with an aspartic acid residue.

50. The Bacillus licheniformis cell of claim 42, wherein the amino acid substitution is with a valine residue.

51. The Bacillus licheniformis cell of claim 43, wherein the amino acid substitution is with an aspartic acid residue.

52. The Bacillus licheniformis cell of claim 43, wherein the amino acid substitution is with a valine residue.

53. The Bacillus licheniformis cell of claim 44, wherein the amino acid substitution is with an aspartic acid residue.

54. The Bacillus licheniformis cell of claim 44, wherein the amino acid substitution is with a valine residue.

55. The Bacillus licheniformis cell of claim 40, wherein
(i) the beta-subunit comprises an amino acid sequence which is at least 90% homologous to the amino acid sequence from position 461 to 500 in SEQ ID NO: 2; and
(ii) the beta-subunit consists of an amino acid substitution at a position corresponding to position 478 in SEQ ID NO: 2.

56. The Bacillus licheniformis cell of claim 41, wherein
(i) the beta-subunit comprises an amino acid sequence which is at least 95% homologous to the amino acid sequence from position 461 to 500 in SEQ ID NO: 2; and
(ii) the beta-subunit consists of an amino acid substitution at a position corresponding to position 478 in SEQ ID NO: 2.

57. The Bacillus licheniformis cell of claim 42, wherein
(i) the beta-subunit comprises an amino acid sequence which is at least 98% homologous to the amino acid sequence from position 461 to 500 in SEQ ID NO: 2; and
(ii) the beta-subunit consists of an amino acid substitution at a position corresponding to position 478 in SEQ ID NO: 2.

58. The Bacillus licheniformis cell of claim 43, wherein
(i) the beta-subunit comprises the amino acid sequence from position 461 to 500 in SEQ ID NO: 2; and
(ii) the beta-subunit consists of an amino acid substitution at a position corresponding to position 478 in SEQ ID NO: 2.

59. The Bacillus licheniformis cell of claim 44, wherein
(i) the beta-subunit comprises the amino acid sequence of SEQ ID NO: 2; and
(ii) the beta-subunit consists of an amino acid substitution at a position corresponding to position 478 in SEQ ID NO: 2.

60. The Bacillus licheniformis cell of claim 45, wherein
(i) the beta-subunit comprises an amino acid sequence which is at least 90% homologous to the amino acid sequence from position 461 to 500 in SEQ ID NO: 2; and
(ii) the beta-subunit consists of an amino acid substitution at a position corresponding to position 478 in SEQ ID NO: 2.

61. The Bacillus licheniformis cell of claim 46, wherein
(i) the beta-subunit comprises an amino acid sequence which is at least 90% homologous to the amino acid sequence from position 461 to 500 in SEQ ID NO: 2; and
(ii) the beta-subunit consists of an amino acid substitution at a position corresponding to position 478 in SEQ ID NO: 2.

62. The Bacillus licheniformis cell of claim 47, wherein
(i) the beta-subunit comprises an amino acid sequence which is at least 95% homologous to the amino acid sequence from position 461 to 500 in SEQ ID NO: 2; and
(ii) the beta-subunit consists of an amino acid substitution at a position corresponding to position 478 in SEQ ID NO: 2.

63. The Bacillus licheniformis cell of claim 48, wherein
(i) the beta-subunit comprises an amino acid sequence which is at least 95% homologous to the amino acid sequence from position 461 to 500 in SEQ ID NO: 2; and
(ii) the beta-subunit consists of an amino acid substitution at a position corresponding to position 478 in SEQ ID NO: 2.

64. The Bacillus licheniformis cell of claim 49, wherein
(i) the beta-subunit comprises an amino acid sequence which is at least 98% homologous to the amino acid sequence from position 461 to 500 in SEQ ID NO: 2; and
(ii) the beta-subunit consists of an amino acid substitution at a position corresponding to position 478 in SEQ ID NO: 2.

65. The Bacillus licheniformis cell of claim 50, wherein
(i) the beta-subunit comprises an amino acid sequence which is at least 98% homologous to the amino acid sequence from position 461 to 500 in SEQ ID NO: 2; and
(ii) the beta-subunit consists of an amino acid substitution at a position corresponding to position 478 in SEQ ID NO: 2.

66. The Bacillus licheniformis cell of claim 51, wherein
(i) the beta-subunit comprises the amino acid sequence from position 461 to 500 in SEQ ID NO: 2; and
(ii) the beta-subunit consists of an amino acid substitution at a position corresponding to position 478 in SEQ ID NO: 2.

67. The Bacillus licheniformis cell of claim 52, wherein
(i) the beta-subunit comprises the amino acid sequence from position 461 to 500 in SEQ ID NO: 2; and
(ii) the beta-subunit consists of an amino acid substitution at a position corresponding to position 478 in SEQ ID NO: 2.

68. The Bacillus licheniformis cell of claim 53, wherein
(i) the beta-subunit comprises the amino acid sequence of SEQ ID NO: 2; and
(ii) the beta-subunit consists of an amino acid substitution at a position corresponding to position 478 in SEQ ID NO: 2.

69. The Bacillus licheniformis cell of claim 54, wherein
(i) the beta-subunit comprises the amino acid sequence of SEQ ID NO: 2; and
(ii) the beta-subunit consists of an amino acid substitution at a position corresponding to position 478 in SEQ ID NO: 2.

70. The Bacillus licheniformis cell of claim 40, wherein the amino acid substitution is A478D or A478V.

71. The Bacillus licheniformis cell of claim 40, wherein the beta-subunit further comprises one or more of the following substitutions:
a substitution at a position corresponding to position 469 in SEQ ID NO: 2;
a substitution at a position corresponding to position 482 in SEQ ID NO: 2;
a substitution at a position corresponding to position 485 in SEQ ID NO: 2; or
a substitution at a position corresponding to position 487 in SEQ ID NO: 2.

72. The Bacillus licheniformis cell of claim 40, which produces a product of a gene which is endogenous to the Bacillus licheniformis cell.

73. The Bacillus licheniformis cell of claim 40, which produces a product of a gene which is exogenous to the Bacillus licheniformis cell.

74. The Bacillus licheniformis cell of claim 40, which produces an enzyme.

75. The Bacillus licheniformis cell of claim 40, which produces an enzyme of a class selected from the group consisting of oxidoreductases, transferases, hydrolases, lyases, isomerases, and ligases.

76. The Bacillus licheniformis cell of claim 40, which produces an enzyme selected from the group consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectinase, peroxidase, phytase, phenoloxidase, polyphenoloxidase, protease, ribonuclease, transferase, transglutaminase, and xylanase.

77. The Bacillus licheniformis cell of claim 40, which produces an amylase.

78. A process for producing a product, comprising cultivating the Bacillus licheniformis cell of claim 40, which comprises a gene encoding the product, in a suitable medium to produce the product.

79. A process for producing a product, comprising cultivating the Bacillus licheniformis cell of claim 41, which comprises a gene encoding the product, in a suitable medium to produce the product.

80. A process for producing a product, comprising cultivating the Bacillus licheniformis cell of claim 42, which comprises a gene encoding the product, in a suitable medium to produce the product.

81. A process for producing a product, comprising cultivating the Bacillus licheniformis cell of claim 43, which comprises a gene encoding the product, in a suitable medium to produce the product.

82. A process for producing a product, comprising cultivating the Bacillus licheniformis cell of claim 44, which comprises a gene encoding the product, in a suitable medium to produce the product.

83. A process for producing a product, comprising cultivating the Bacillus licheniformis cell of claim 45, which comprises a gene encoding the product, in a suitable medium to produce the product.

84. A process for producing a product, comprising cultivating the Bacillus licheniformis cell of claim 46, which comprises a gene encoding the product, in a suitable medium to produce the product.

85. A process for producing a product, comprising cultivating the Bacillus licheniformis cell of claim 47, which comprises a gene encoding the product, in a suitable medium to produce the product.

86. A process for producing a product, comprising cultivating the Bacillus licheniformis cell of claim 48, which comprises a gene encoding the product, in a suitable medium to produce the product.

87. A process for producing a product, comprising cultivating the Bacillus licheniformis cell of claim 49, which comprises a gene encoding the product, in a suitable medium to produce the product.

88. A process for producing a product, comprising cultivating the Bacillus licheniformis cell of claim 50, which comprises a gene encoding the product, in a suitable medium to produce the product.

89. A process for producing a product, comprising cultivating the Bacillus licheniformis cell of claim 51, which comprises a gene encoding the product, in a suitable medium to produce the product.

90. A process for producing a product, comprising cultivating the Bacillus licheniformis cell of claim 52, which comprises a gene encoding the product, in a suitable medium to produce the product.

91. A process for producing a product, comprising cultivating the Bacillus licheniformis cell of claim 53, which comprises a gene encoding the product, in a suitable medium to produce the product.

92. A process for producing a product, comprising cultivating the Bacillus licheniformis cell of claim 54, which comprises a gene encoding the product, in a suitable medium to produce the product.

93. A process for producing a product, comprising cultivating the Bacillus licheniformis cell of claim 55, which comprises a gene encoding the product, in a suitable medium to produce the product.

94. A process for producing a product, comprising cultivating the Bacillus licheniformis cell of claim 56, which comprises a gene encoding the product, in a suitable medium to produce the product.

95. A process for producing a product, comprising cultivating the Bacillus licheniformis cell of claim 57, which comprises a gene encoding the product, in a suitable medium to produce the product.

96. A process for producing a product, comprising cultivating the Bacillus licheniformis cell of claim 58, which comprises a gene encoding the product, in a suitable medium to produce the product.

97. A process for producing a product, comprising cultivating the Bacillus licheniformis cell of claim 59, which comprises a gene encoding the product, in a suitable medium to produce the product.

98. A process for producing a product, comprising cultivating the Bacillus licheniformis cell of claim 60, which comprises a gene encoding the product, in a suitable medium to produce the product.

99. A process for producing a product, comprising cultivating the Bacillus licheniformis cell of claim 61, which comprises a gene encoding the product, in a suitable medium to produce the product.

100. A process for producing a product, comprising cultivating the Bacillus licheniformis cell of claim 62, which comprises a gene encoding the product, in a suitable medium to produce the product.

101. A process for producing a product, comprising cultivating the Bacillus licheniformis cell of claim 63, which comprises a gene encoding the product, in a suitable medium to produce the product.

102. A process for producing a product, comprising cultivating the Bacillus licheniformis cell of claim 64, which comprises a gene encoding the product, in a suitable medium to produce the product.

103. A process for producing a product, comprising cultivating the Bacillus licheniformis cell of claim 65, which comprises a gene encoding the product, in a suitable medium to produce the product.

104. A process for producing a product, comprising cultivating the Bacillus licheniformis cell of claim 66, which comprises a gene encoding the product, in a suitable medium to produce the product.

105. A process for producing a product, comprising cultivating the Bacillus licheniformis cell of claim 67, which comprises a gene encoding the product, in a suitable medium to produce the product.

106. A process for producing a product, comprising cultivating the Bacillus licheniformis cell of claim 68, which comprises a gene encoding the product, in a suitable medium to produce the product.

107. A process for producing a product, comprising cultivating the Bacillus licheniformis cell of claim 69, which comprises a gene encoding the product, in a suitable medium to produce the product.

108. A process for producing a product, comprising cultivating the Bacillus licheniformis cell of claim 70, which comprises a gene encoding the product, in a suitable medium to produce the product.

109. A process for producing a product, comprising cultivating the Bacillus licheniformis cell of claim 71, which comprises a gene encoding the product, in a suitable medium to produce the product.

110. A process for producing an endogenous product, comprising cultivating the Bacillus licheniformis cell of claim 72, which comprises a gene encoding the endogenous product, in a suitable medium to produce the endogenous product.

111. A process for producing an exogenous product, comprising cultivating the Bacillus licheniformis cell of claim 73, which comprises a gene encoding the exogenous product, in a suitable medium to produce the exogenous product.

112. A process for producing an enzyme, comprising cultivating the Bacillus licheniformis cell of claim 74, which comprises a gene encoding the enzyme, in a suitable medium to produce the product.

113. A process for producing an enzyme, comprising cultivating the Bacillus licheniformis cell of claim 75, which comprises a gene encoding the enzyme, in a suitable medium to produce the enzyme.

114. A process for producing an enzyme, comprising cultivating the Bacillus licheniformis cell of claim 76, which comprises a gene encoding the enzyme, in a suitable medium to produce the enzyme.

115. A process for producing an amylase, comprising cultivating the Bacillus licheniformis cell of claim 77, which comprises a gene encoding the amylase, in a suitable medium to produce the amylase.

116. An isolated Bacillus cell comprising a nucleic acid sequence encoding an RNA-polymerase, wherein
(a) the RNA-polymerase comprises a beta-subunit which comprises
(i) an amino acid sequence which is at least 90% homologous to the amino acid sequence from position 461 to 500 in SEQ ID NO: 2; and
(ii) an amino acid substitution at a position corresponding to position 478 in SEQ ID NO: 2; and
(b) the RNA-polymerase has RNA-polymerase activity.

117. The Bacillus cell of claim 116, which is selected from the group consisting of Bacillus alkalophilus, Bacillus amyloliquefaciens, Bacillus brevis, Bacillus circulans, Bacillus clausii, Bacillus coagulans, Bacillus lautus, Bacillus lentus, Bacillus megaterium, Bacillus stearothermophilus, Bacillus subtilis, and Bacillus thuringiensis.

118. The Bacillus cell of claim 116, which is a Bacillus amyloliquefaciens cell.

119. The Bacillus cell of claim 116, which is a Bacillus clausii cell.

120. The Bacillus cell of claim 116, which is a Bacillus lentus cell.

121. The Bacillus cell of claim 116, which is a Bacillus stearothermophilus cell.

122. The Bacillus cell of claim 116, which is a Bacillus subtilis cell.

123. A process for producing a product, comprising cultivating the Bacillus cell of claim 116, which comprises a gene encoding the product, in a suitable medium to produce the product.

124. A process for producing a product, comprising cultivating the Bacillus cell of claim 117, which comprises a gene encoding the product, in a suitable medium to produce the product.

125. A process for producing a product, comprising cultivating the Bacillus cell of claim 118, which comprises a gene encoding the product, in a suitable medium to produce the product.

126. A process for producing a product, comprising cultivating the Bacillus cell of claim 119, which comprises a gene encoding the product, in a suitable medium to produce the product.

127. A process for producing a product, comprising cultivating the Bacillus cell of claim 120, which comprises a gene encoding the product, in a suitable medium to produce the product.

128. A process for producing a product, comprising cultivating the Bacillus cell of claim 121, which comprises a gene encoding the product, in a suitable medium to produce the product.

129. A process for producing a product, comprising cultivating the Bacillus cell of claim 122, which comprises a gene encoding the product, in a suitable medium to produce the product.
